(19) United States
(12) Patent Application Publication
(10) Pub. No.: US 2019/0122073 A1
(43) Pub. Date: Apr. 25, 2019

(54) SYSTEM AND METHOD FOR QUANTIFYING UNCERTAINTY IN REASONING ABOUT 2D AND 3D SPATIAL FEATURES WITH A COMPUTER MACHINE LEARNING ARCHITECTURE

(71) Applicant: The Charles Stark Draper Laboratory, Inc., Cambridge, MA (US)

(72) Inventors: Onur Ozdemir, Arlington, MA (US); Benjamin Woodward, Medford, MA (US); Andrew A. Berlin, Lexington, MA (US)

(21) Appl. No.: 15/790,332

(22) Filed: Oct. 23, 2017

(51) Int. Cl. G06K 9/62; G06N 99/00; G16H 30/40; G06N 3/08; G06K 9/00; G06T 7/00; G06T 7/143; G06K 9/78; A61B 5/055; A61B 5/00; A61B 6/03; A61B 8/08; A61B 6/00

(52) U.S. Cl. CPC G06K 9/6256; G06T 7/248; G16H 30/40; G06N 3/08; G06K 9/00791; G06K 9/6267; G06T 7/0012; G06T 7/143; G06K 9/6288; G06K 9/0063; G06K 9/78; A61B 5/055; A61B 5/7267; A61B 6/032; A61B 8/085; A61B 8/481; A61B 5/7282; A61B 6/5217; A61B 8/5223; G06T 2207/10072; G06T 2207/30096; G06T 2207/10032; G06T 2207/30252; G06N 99/005

(57) ABSTRACT

This invention provides a system and method to propagate uncertainty information in a deep learning pipeline. It allows for the propagation of uncertainty information from one deep learning model to the next by fusing model uncertainty with the original imagery dataset. This approach results in a deep learning architecture where the output of the system contains not only the prediction, but also the model uncertainty information associated with that prediction. The embodiments herein improve upon existing deep learning-based models (CADe models) by providing the model with uncertainty/confidence information associated with (e.g. CADe) decisions. This uncertainty information can be employed in various ways, including (a) transmitting uncertainty from a first stage (or subsystem) of the machine learning system into a next (second) stage (or the next subsystem), and (b) providing uncertainty information to the end user in a manner that characterizes the uncertainty of the overall machine learning model.

### FIELD OF THE INVENTION

This invention relates to machine learning systems and methods, and more particularly to application of machine learning in image analysis and data analytics to identify features of interest.

### BACKGROUND OF THE INVENTION

Uncertainty modeling for reasoning about two-dimensional (2D) and three-dimensional (3D) spaces using machine learning can be challenging. This modeling technique can be employed in a variety of applications, such as computer vision, which require spatial interpretation of an imaged scene. Machine learning (ML) is ubiquitous in computer vision. Most ML techniques fall into two broad categories: (a) traditional ML techniques that rely on hand-engineered image features, and (b) deep learning techniques that automatically learn task-specific useful features from the raw image data. The second category (i.e. deep learning) has consistently outperformed the first category in many computer vision applications in recent years. Despite the success of deep learning models in computer vision, such are essentially black-box systems lacking predictive/model uncertainty, which hinder the adoption of these models in actual clinical settings where ML predictions could potentially be used to guide serious (even life and death) decisions. Thus, it is desirable to develop techniques for effectively introducing uncertainty models into the overall deep learning model.

An area where deep learning can provide a valuable tool is in the field of computer-aided detection (CADe) of pulmonary nodules using low-dose CT scans. In addition to CADe, other medical imaging modalities (such as MM, ultrasound, and PET), as well as other imagery reasoning applications such as medical anomaly detection, object/target detection using RADAR/LIDAR/SONAR data acquired by various different imaging platforms (e.g. satellite/aerial imaging platforms) can benefit from deep learning modelling. In addition, non-image data that has a spatial or temporal component, such as weather records, social media activity types, and supply chain activity can benefit from a deep learning approach to data analysis.

By way of useful background, computer-aided detection (CADe), as well as computer-aided diagnosis (CADx), are systems that assist medical practitioners and others in the interpretation of medical images. Imaging techniques in X-ray, MRI, and ultrasound diagnostics yield a great deal of information that the radiologist or other medical professional must analyze and evaluate in a timely manner. CAD systems process digital images for typical appearances and to highlight conspicuous sections, such as possible diseases, in order to offer input to support a decision taken by the practitioner. CADe is an interdisciplinary technology combining elements of artificial intelligence and computer vision with radiological and pathology image processing. A typical application is the detection of an internal tumor or lesion (e.g. diagnosis of breast cancer, the detection of polyps in the colon, and lung cancer).

CADe, and CAD in general, is fundamentally based on complex pattern recognition algorithms. X-ray or other types of images are scanned for suspicious structures. Normally a few thousand images are required to optimize the algorithm. Digital image data are copied to a CAD server in an appropriate data format (e.g. DICOM), and are prepared and analyzed in several steps. These steps include: (a) preprocessing for reduction of artifacts, image noise reduction, leveling (harmonization) of image quality (increased contrast) for clearing the image parameters (e.g. different exposure settings), and filtering; (b) segmentation for differentiation of different structures in the image (e.g. heart, lung, ribcage, blood vessels, possible round lesions, matching with anatomic database, and sample gray-values in volume of interest); (c) structure/ROI (Region of Interest) analysis, in which a detected region is analyzed individually for special characteristics, which can include compactness, form, size and location, reference to close-by structures/ROIs, average grey level value analysis within the ROI, and proportion of grey levels to the border of the structure inside the ROI; and (d) Evaluation/classification of the structure, which is analyzed so that each ROI is evaluated individually (scoring). Some examples of classification algorithms include, but are not limited to, (i) nearest-neighbor rule (e.g. k nearest neighbors), (ii) minimum distance classifier cascade classifier, (iii) naive Bayesian classifier, (iv) artificial neural network, radial basis function network (RBF), and/or (v) support vector machine (SVM). Based on these procedure steps, if the detected structures within the image(s) have reached a certain threshold level, they are highlighted in the image for the practitioner to study.

In the particular example of application of CADe to lung cancer, it is recognized that this disease is one of the most deadly human cancers, and early diagnosis is the key to reduce mortality. Early detection of pulmonary nodules is crucial for early diagnosis of lung cancer. CADe of pulmonary nodules using low-dose computed tomography (CT) for high-risk individuals has become an active area of research, especially due to the development of advanced deep/machine learning techniques combined with the availability of computation powered by graphics processing units (GPUs), or other similar devices employing (e.g.) STMD architecture, etc., and the development of modern machine learning software libraries. CADe using ML has the potential to discover patterns in large scale datasets, which would be highly difficult or impossible for humans to manually analyze, while eliminating inter-operator variability. Notably, it is common for different radiologists to form widely different conclusions on the same set of images.

However, as described above, the absence of uncertainty estimation, modeling, and/or tracking in the techniques applied to such CADe analysis render it less reliable and useful in a fully automated scenario where the practitioner takes a less active role in analyzing the image data and relies more on the results of the computer-based analysis thereof. Hence, the current approach lacks needed confidence, which must be provided by the human user's judgment.

### SUMMARY OF THE INVENTION

This invention overcomes disadvantages of the prior art by providing a system and method that employs a novel technique to propagate uncertainty information in a deep learning pipeline. Advantageously, the illustrative system and method allows for the propagation of uncertainty information from one deep learning model to the next by fusing model uncertainty with the original imagery dataset. This approach results in a deep learning architecture where the output of the system contains not only the prediction, but also the model uncertainty information associated with that prediction. More particularly, the embodiments herein improve upon existing deep learning-based models (e.g. CADe models) by providing the model with uncertainty/confidence information associated with (e.g. CADe) decisions. This uncertainty information can be employed in various ways, two of which are, (a) transmitting uncertainty from a first stage (or subsystem) of the machine learning system into a next (second) stage (or the next subsystem), and (b) providing uncertainty information to the end user in a manner that characterizes the uncertainty of the overall machine learning model. In other words, existing models do not provide estimates of confidence, which is extremely important for critical decision making applications such as cancer detection. In an exemplary embodiment (the general teachings of which are applicable to a broad range of potential applications), the system and method herein addresses the problem of CADe analysis of pulmonary nodules and improves upon present techniques by advantageously and uniquely integrating model confidence into deep learning models. The resulting system and method develops a high-performing CADe model for automatic detection of pulmonary nodules and related structures within 2D and 3D image data, and provides model confidence (or uncertainty information) associated with the CADe decisions for the model so as to make the overall CADe system more interpretable and easier to be adopted by practitioners (e.g. doctors and radiologists) in clinical settings.

In an illustrative embodiment, this invention provides a system and method for detecting and/or characterizing a property of interest in a multi-dimensional space. The system and method receives a signal based upon acquired data from a subject or object in the multi-dimensional space. It interprets a combination of information from the signal (e.g. signal intensity, signal phase, etc.) and confidence information (which quantifies uncertainty), and based thereon, performs at least one of detection and characterization of at least one property of interest related to the object or subject. Illustratively, the multi-dimensional space can be a 2D image (such as pixels) or a 3D spatial representation (such as voxels, multi-layer slices, etc.). The detection and/and characterization can include use of a learning algorithm (such as a convolutional neural network) trained based on the combination of information from the signal and confidence level information, and can optionally include evaluation by the learning algorithm that has been trained via (e.g.) the convolutional neural network. Illustratively, the system and method can include estimating the confidence level based upon uncertainty using dimensional representations of a lower dimension (e.g. 2D image slices) than the (higher) multi-dimensional space (such as a 3D CT scan), in which at least two estimates of uncertainty, which are based on the dimensional representations of the lower dimension, are assembled to form a representation of the uncertainty in the multi-dimensional space (for example, computing uncertainty for 2D slices, and then stacking those uncertainties to fill 3D space). The step of estimating the confidence level can include using additional image channels to represent each of a plurality of confidence levels.

Additionally, the confidence level can be represented by at least one of a sparse representation and a hierarchical representation in order to reduce the overall amount of data, as a form of compression. More particularly, the confidence level can be represented by at least one of a quadtree (e.g. for two-dimensions), an octree (e.g. for three dimensions), a multi-scale image representation, and a phase representation. The acquired data can vehicle sensor data, including at least one of LIDAR, RADAR and ultrasound, which characterizes at least one object in images to evaluate, including at least one of (a) obstacles to avoid, (b) street signs to identify, (c) traffic signals, (d) road markings, and/or (e) other driving hazards. Illustratively, the vehicle sensing data can be used for controlling an action or operation of a device of a land vehicle, aircraft or watercraft based on an object classifier that reports low confidence level in a classification thereof.

In another embodiment, the acquired data is medical image data, including at least one of CT scan images, MM images, or targeted contrast ultrasound images of human tissue, and the property of interest is a potentially cancerous lesion. Illustratively, the detection and characterization is diagnosis of a disease type, and the information from the signal is one or more suspected lesion location regions-of-interest, and the confidence levels are associated with each region-of-interest that is suspected. In embodiments, the steps of receiving, interpreting and performing are performed in association with a deep learning network that defines a U-net style architecture. The deep learning network can also incorporate a Bayesian machine learning network. In another embodiment, the acquired data is received by an ad-hoc sensor network, in which a network configuration (i.e. tasking) is reconfigured so as to optimize the confidence level in a detected parameter. This sensor network can include a network of acoustic sensors in which a local data fusion (i.e. communication) is adjusted based on a confidence of detection of a property of interest in the signal thereof. Illustratively, the confidence level can be associated with the signal to enhance performance by using a thresholding step to eliminate low confidence results. The signal can also be based on aerial acquisition and the property of interest is related to a sea surface anomaly, an aerial property or an air vehicle. In embodiments, the confidence level related to the subject is classified by machine learning networks to an end-user (human or machine/processor), including a spatial indicator that augments an ordinary intensity of the signal in a manner that conveys certainty. In embodiments, the system and method can include fusing uncertainty information temporally across multiple image frames derived from the signal to refine an estimate of the confidence level. The step of fusing can be based on at least one of tracking of the subject and spatial location of the subject, and/or the step of fusing can include (a) taking a MAXIMUM across multiple time points (b) taking a MINIMUM across multiple time points, (c) taking a MEAN across multiple time points, and (d) rejecting extreme outliers across multiple time points.

In a further illustrative embodiment, a system that overcomes limitations of uncertainty measurement is provided. This system includes a morphological filter that adjusts a confidence level associated with a region based on confidence levels associated with neighbors of the region.

In another embodiment a system and method for acquiring one or more images to be scanned for presence of a property of interest is provided. This system and method acquires a first set of images, analyzes the first set of images to detect the property of interest and a confidence level associated with the detection; and iteratively adjusts at least one image acquisition parameter (e.g. camera focus, exposure time, radar power level, frame rate, etc.) in a manner that optimizes or enhances the confidence level associated with the detection of the property of interest.

In another embodiment, a system for detecting a property of interest in a sequence of acquired images is provided, in which at least one of a plurality of available image interpretation parameters (such as image thresholding levels, image pre-processing parameters—including, but not limited to multi-pixel fusion, image smoothing parameters, contrast enhancement parameters, image sharpening parameters—machine learning decision-making thresholds, etc.) is iteratively adjusted by a processor so as to optimize a confidence level in detection of the property of interest. The image interpretation parameters can include at least one of image thresholding levels, image pre-processing parameters, multi-pixel fusion, image smoothing parameters, contrast enhancement parameters, image sharpening parameters, and machine learning decision-making thresholds.

In yet another embodiment, a system for utilizing a conventionally trained neural network, which is free-of training using confidence level data to analyze signal data is provided. The data has been augmented by confidence level, in which the signal data is weighted based on confidence prior to presentation to the conventionally-trained neural network. The conventionally trained neural network can comprise a tumor lesion characterizer.

### BRIEF DESCRIPTION OF THE DRAWINGS

The invention description below refers to the accompanying drawings, of which:

FIG. 1 is a diagram of a generalized system in which image (or similar form(s) of) data is acquired from an object or subject of interest using an imaging medium (e.g. visible or nonvisible electromagnetic radiation/visible light, etc.), and transmitted to a processor that analyzes the data in accordance with the illustrative system and method;

FIG. 2 is a block diagram showing a data flow in a method for image uncertainty propagation and fusion through a cascade of Bayesian neural networks, which can be used for lesion detection and/or other tasks according to embodiments herein;

FIG. 3 is a flow diagram of an overall system and method for use in performing CADe and other related forms of image analysis using Bayesian neural networks with uncertainty computed in accordance with embodiments herein;

FIG. 4 is a flow diagram showing an exemplary procedure for segmenting image data as part of a localization process in the system and method of FIG. 3;

FIG. 5 is a flow diagram of a procedure for generating multi-channel image data with respect to original image data within a Bayesian inference procedure of the segmentation process of FIG. 4;

FIG. 6 is a diagram of exemplary acquired (test) image data showing various stages of segmentation according to the procedure of FIG. 5;

FIGS. 7 and 8 are diagrams of an exemplary image data from FIG. 6 showing the results of the operation of the Bayesian neural network to remove false positives;

FIG. 9 is a flow diagram showing a procedure for extracting 3D voxels as part of a post-processing procedure according to the overall procedure of FIG. 5;

FIG. 10 is an exemplary 3D convolutional neural network (3D CNN) architecture for pulmonary nodule identification according to the overall procedure of FIG. 5;

FIG. 11 is a graph showing plots of true positives versus false positives for detection of exemplary nodules for 3D CNNs of FIG. 10, employing different kernel sizes and an ensemble technique;

FIG. 12 is a graph showing a plot of true positives versus false positives for detection of exemplary nodules for one of the 3D CNNs of FIG. 11 and with the addition of a merging of the original CT image with the Bayesian segmentation network output images (mean and variance images), normalization the voxel intensity values, and running of this merged structure through one of the 3D CNNs;

FIG. 13 is a diagram of an exemplary CADe graphical user interface (GUI) display showing an image of the region of interest (a lung) and an associated low confidence nodule detection result, which therefore includes a prompt to the user (e.g. a radiology technician) to seek expert (e.g. a radiologist) opinion in further diagnosis;

FIG. 14 is a diagram of an exemplary CADe graphical user interface (GUI) display showing an image of the region of interest (a lung) and an associated high confidence nodule detection result, which therefore includes a includes a prompt to the user (e.g. radiologist or other appropriate practitioner) to investigate further on a high-priority basis; and

FIG. 15 is a diagram of a vehicle-borne LIDAR (or similar imaging system) for use in controlling the vehicle and/or advising the user/driver that employs object detection and characterization processes in accordance with the embodiments herein.

### DETAILED DESCRIPTION

### I. System Overview

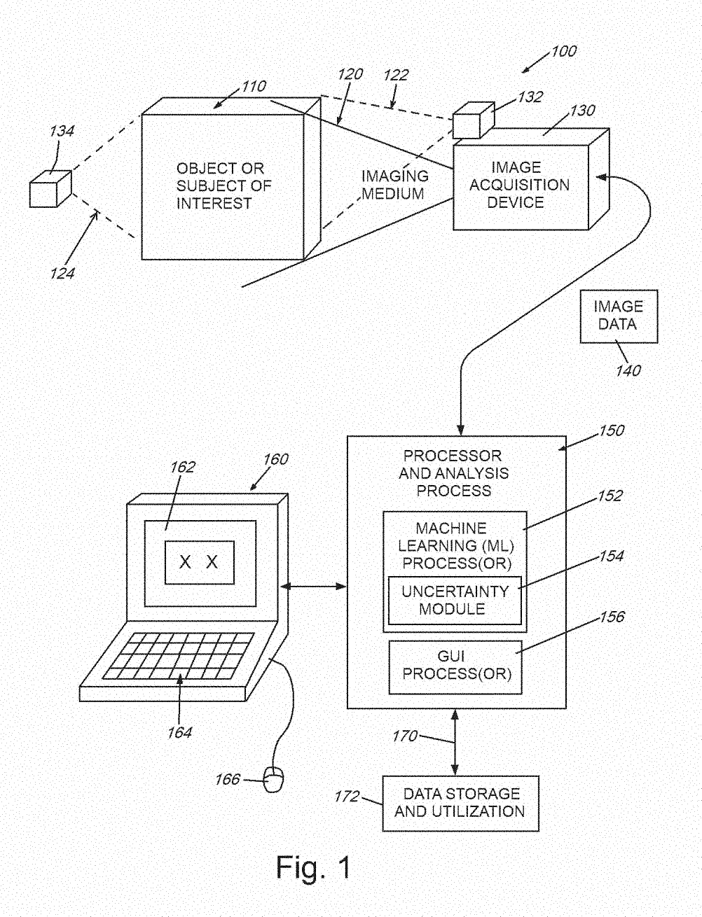

FIG. 1 is a diagram showing a generalized arrangement 100 for acquiring and analyzing image (and other related) data in 2D or 3D space. An object or other appropriate subject of interest 110 is located within a scene from which meaningful information is to be extracted. In the case of medical imaging, the object 110 can be all or a portion of a (e.g. human) body. The imaging medium can be electromagnetic radiation, such as X-rays, ultrasound waves, or various electromagnetic fields (for example MRI-generated fields). The medium can also be visible, or near visible light. More generally, the medium can be any type, or combination of types, of information-carrying transmissions including, but not limited to those used in automotive, aerospace and marine applications (e.g. navigation, surveillance and mapping)—for example, radio waves, SONAR, RADAR, LIDAR, and others known to those of skill. The appropriate image acquisition device 130—for example a device (receiver) that receives external natural and man-made emissions—can be employed to convert emissions into a meaningful data form. The receiver can be paired with one or more emitters/transmitters 132, 134 that generate appropriate imaging medium/media (dashed lines 122, 124). Hence, the receiver 130 can rely upon reflections from the object (emitter 132 and medium 122), in the case of (e.g.) near-visible and visible light, RADAR, SONAR, LIDAR, or can rely upon transmittance through the object (emitter 134 and medium 124) in the case of (e.g.) certain forms of ultrasound or vibration, heat, electromagnetic fields, X-rays, neutron beams, etc. Alternatively, in the case of various naturally occurring image medium sources—e.g. visible light, cosmic rays, ocean and airborne sound, seismic waves, the transmitter is typically nature—although in some instances, a man-made source can be included in such media, such as a hydrophone, speaker or vibration-generator. In yet other embodiments, the medium 122, 124 can be characterized as emission from the subject/object itself, based on appropriate stimulus for the transmitter (132/134). For example, it is expressly contemplated that the medium can be energy emitted by a (e.g.) Positron Emission Tomography (PET) scan tracer particle, or photons emitted due to optically or electrically excited molecules, as occurs in Raman spectroscopy. All forms of electromagnetic, particle and/or photonic energy can characterize the medium measured herein and from which imagery or similar datasets are derived by the acquisition device.

The image acquisition device generates a 2D or 3D map of the received medium 120 with respect to the object/subject 110. This can be represented as an array of 2D pixels, 3D voxels, or another acceptable form having a predetermined resolution and range of intensity values. The data (termed generally “image data”) 140 is transmitted to a processor and associated analysis process 150 in accordance with the embodiments herein. The image data can be preprocessed as appropriate to include edge information, blobs, etc. (for example based on image analysis conducted using appropriate, commercially available machine vision tools). The image data can also be presented in a form native to certain types of devices—for example a 3D rendering of a body formed from slices in the case of an X-ray-based computerized tomography (CT) scan. The processor 150 can be integrated in whole or in part within the acquisition device 130, or can be a separate platform—for example one that is instantiated in hardware and/or software (consisting of non-transitory program instructions)—such as a standalone, PC, server, laptop, tablet or handheld computing device 160. More generally the processor 150 communicates with such a device 160 so as to provide an appropriate interface (e.g. a graphical user interface (GUI)) that can include a display and/or touchscreen 162 and, where applicable, other manual interface functions, such as a keyboard 164 and cursor-actuation device/mouse 166. The computing device 160 and/or processor can be networked via any appropriate wired or wireless link 170 to external devices and data stores 172—such as those found locally and/or on the cloud via the well-known Internet.

The processor 150 can be organized in any appropriate way using any appropriate hardware and software. For purposes of the description, the processor includes various functional processes or modules (in hardware, software or both), including a machine learning (ML) process(or) 152. This process(or) analyzes the data for certain desired feature information—potentially based on trained models. An uncertainty (functional) module 154 is provided within the overall ML process(or) 152, as described in detail below, and provides uncertainty computations to the ML process(or). A GUI process(or) 156 organizes and displays (or otherwise presents (e.g. for storage)) the analyzed data results in a graphical and/or textual format for a user to employ in performing a related task. Other functional processes and/or modules can be provided as appropriate for the type of data and desired output of results.

### II. Bayesian Deep Learning Model for Use in Image Analysis

A. Image Segmentation and Concatenation

As described above, the generalized image acquisition and processor architecture presented herein is applicable to a broad range of possible uses in various types of image and data analysis. By way of non-limiting example, the following description, which can be applicable to various data types and systems, and is described particularly in a CADe environment related to acquired CT scan imagery. More particularly, the exemplary images and processing, shown and described herein, analyze thoracic CT scans of a patent's lung, which contains a cancerous lesion. In analyzing imagery, the system and method herein employs a Bayesian deep learning model, examples of which include convolutional neural networks, recurrent neural networks, and feedforward neural networks. In Bayesian deep learning models, the parameters (or weights) of the network are random variables, as opposed to deterministic variables as in standard neural networks. As a result, the network can model epistemic uncertainty—i.e. model uncertainty about the predictions resulting from ambiguity or sufficiency (or both) in training data. Hence, this addresses a scenario in which the training data is self-inconsistent, as opposed to simply lacking in quantity, or where the model has low confidence because it simply has not been exposed to a sufficient number of similar examples. The output of a Bayesian neural network (the prediction given the input) is a random variable represented as a conditional probability distribution p(y|x), where y and x are the output and the input of the network, respectively. In other words, this conditional distribution encodes complete information about the prediction, encapsulating its uncertainty, which could be represented as the variance or the entropy of the distribution. This distribution also allows the process to extract higher order moments or any other higher order statistics, if desired. In a machine learning pipeline where there occurs a cascade of multiple machine learning models, existing solutions do not provide a way to propagate uncertainty information. This disadvantage is addressed by obtaining the statistics from p(y|x) and using these statistics as inputs into the next machine learning model (in addition to the original input) within the pipeline. As described further below, this process not only improves the performance of the overall system by propagating and fusing uncertainty from multiple models, but also results in a final prediction output that accurately models epistemic uncertainty, thereby providing the end user with an estimate of model confidence about the prediction.

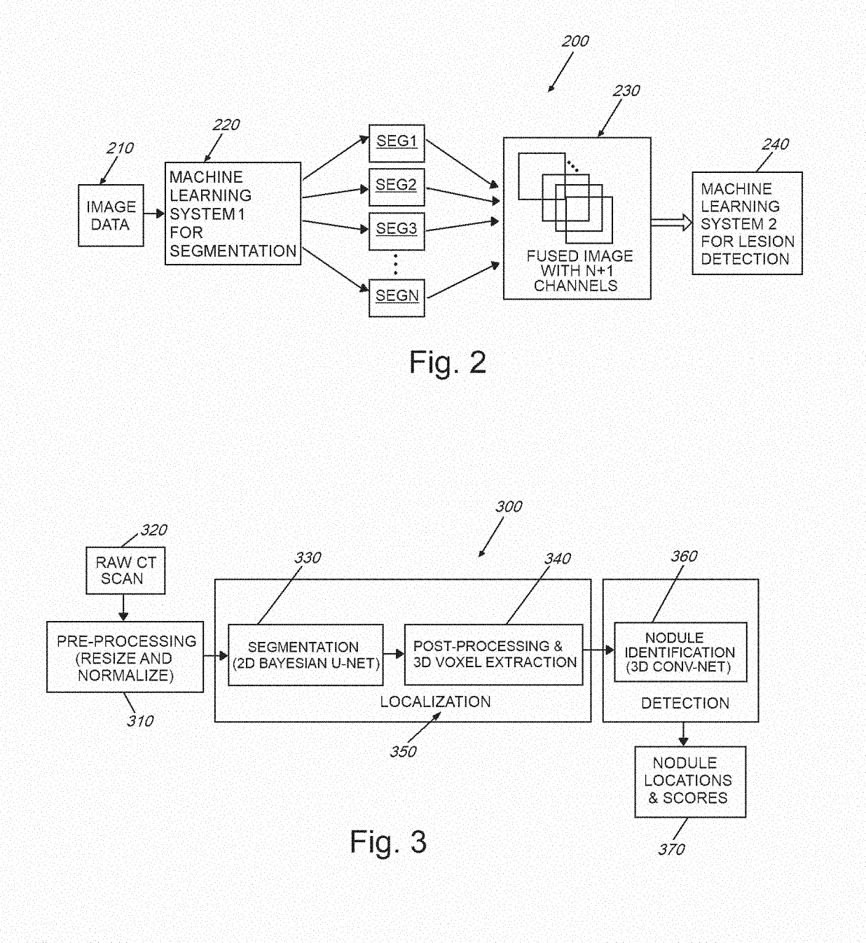

A specific application of the deep learning model is shown by way of example in the diagram 200 of FIG. 2 (e.g. for use in a medical anomalous lesion detection problem using imagery data 210). A standard machine learning approach to this problem is to first segment the image (using depicted Machine Learning System 1 (220)) into the depicted image segments (SEG1-SEGN) to narrow the search space, and then to perform detection (Machine Learning System 2 (240) in the pipeline) within each image region. In the depicted example, image segmentation is performed by classifying each pixel value into one of N regions. In standard machine learning-based segmentation, these classification decisions are deterministic for a given input image. In the illustrative model, the segmentation decisions are probabilistic, i.e., there is a complete distribution of segmentation decisions/outputs. The procedure, hence, generates a series of statistics images, each of which represents a particular statistic (for example the first and the second order moments, (i.e.) mean and variance of the confidence in the classification result) computed from pixel-wise segmentation probability distributions. Optionally, these statistics images are fused/concatenated with the original image to create a composite image 230 consisting of the original image and the statistics obtained from the segmentation networks, essentially treating each statistics image as a new channel in the composite image. This composite image is then provided to the second neural network 240 to perform final lesion detection.

It should be clear that the novel system and method herein effectively (and uniquely) propagates uncertainty information from first neural network to a second neural network (whether that information is embedded as pixels within an image or by another modality). More particularly, the second network is trained independently of the first network. Additionally, the system and method is arranged so that at least one network (typically, the second network) receives as an input, uncertainty information during the training process, as well as during the evaluation process.

Concatenating statistics images from the new channels with the original image to create a new multi-channel composite image 230 and providing this new composite image into the next network 240 for final detection are novel aspects of the illustrative system and method. In an embodiment, the second network 240 is implemented as a Bayesian neural network, which outputs the prediction as a conditional probability distribution. In other words, a cascade of Bayesian neural networks (segmentation and detection networks) fuse uncertainty information from one network to the next until a final prediction output is obtained (e.g. as a probability distribution).

The following description includes image data from an actual medical condition and patient to which the illustrative system and method is applied—more particularly, the challenge of performing CADe of pulmonary nodules, and use of a two-stage Bayesian convolutional neural network therewith.

B. Two-Stage Convolutional Neural Networks for Use in (e.g.) Pulmonary Nodule Detection

FIG. 3 shows a diagram 300 that depicts four functional modules/processes of the system and method used to detect (e.g.) pulmonary nodules. Although the specific example herein relates to CT scans used to detect internal (e.g. cancerous) nodules and lesions, it is expressly contemplated that the terms “nodule” or “lesion” can be substituted with a more generalized “feature of interest” to be detected, located and scored within respect to an object or subject and that the exemplary CT scan image data can be substituted for any image data that relays 2D and 3D information about a remote object or subject. The modules/processes of the system and method are now described in further detail, and include the following:

1. Pre-processing 310, in which each input raw (e.g.) CT image 320 is resampled (by way of example) to an isotropic 1 mm³ resolution. Then, each image is either cropped or padded on an axial plane to derive a uniform size of 512×512×N_{z}, where N_{z} is the number of scans (slices) along the z axis (since the imagery is characterized as a 3D spatial representation of the patient's pulmonary region) in the image coordinate space. Before segmentation, axial image slices are extracted for each scan, and then their respective intensity values are each normalized to equal zero (0) mean and unit variance across all images—such images in this example being characterized as “training images” in that they are employed to train the neural network for subsequent runtime analysis operations.

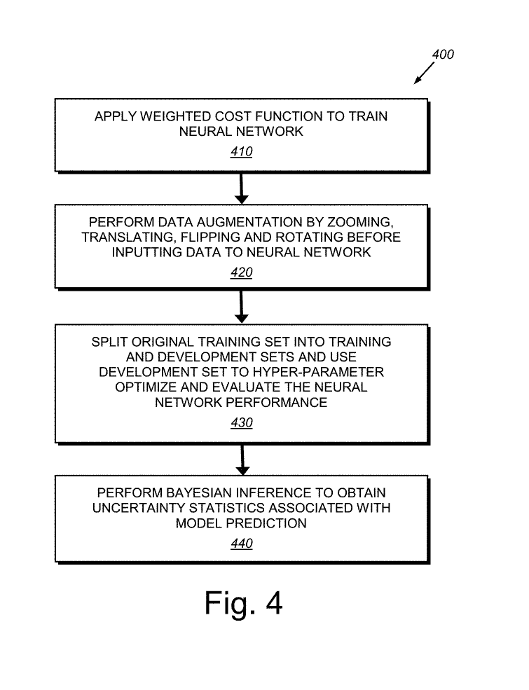

2. Segmentation 330, in which the neural network architecture 200 described in FIG. 2 above, is a convolutional neural network developed specifically for image segmentation tasks. The embodiments herein can implement various improvements and updated design choices relative to a traditional U-net framework (in terms of both training, evaluation, and prediction tasks) to enhance its performance for the exemplary task of segmenting lung nodules. The U-net is a particular type of convolutional neural network architecture that can be employed according to an illustrative embodiment. However, alternate embodiments can employ alternate versions of a neural network architecture that should be clear to those of skill. Notably, the embodiment implements a Bayesian version of U-net, which enables the generation of stochastic outputs from the network, and therefore, effectively quantifies the uncertainty associated with segmentation decisions. The use of a Bayesian U-net affords a novel approach for pulmonary nodule segmentation, and/or any other task. In an exemplary arrangement, a 10-layer U-net is employed for the task. The following design considerations are addressed by the exemplary embodiment, and with further reference to the procedure 400 of FIG. 4:

(a) Training cost function adjustment (step 410): Since an exemplary goal is to detect pulmonary nodules that are very small (3 mm³-25 mm³) compared to the original CT scan size, a weighted cost function is employed for training the neural network. This approach de-biases the network from learning only the background pixels that have significantly higher occurrence frequency than that of nodule pixels. Our cost function per-batch is a weighted cross-entropy which is expressed as:

$${ℒ}_{B} ( θ ) = {\sum }_{B} {\sum }_{x} y ( x ) w ( x ) log \hat{y} ( x ; θ ) + ( 1 - y ( x ) ) ( 1 - w ( x ) ) log ( 1 - \hat{y} ( x ; θ ) )$$

where x denotes pixel position on an axial slice, y(x)∈{0, 1} denotes whether pixel x is a nodule (1) or not (0), w(x)∈[0, 1] is the weight representing the contribution of cross-entropy loss associated with pixel x, ŷ(x; θ)∈[0, 1] is the output of the network for pixel x denoting the probability that pixel x is a nodule (parameterized by θ), θ is the vector of network weights that are learned via training (i.e. by minimizing the loss function L(θ)), and B is the set of slices in a training batch. Note that the weight terms w(x) allow the use of different cross-entropy contributions for different pixels in the loss function, which in turn, encourages the network to make fewer errors for larger weighted pixels and vice versa. It has been recognized that w(x)=0.9 or 0.85 results in desirable optimization of true positive and false positive rates. This contrasts traditional approaches proposed in the literature where w(x) is set to be the inverse frequency of the target pixels normalized to 1. Such a value would correspond to approximately 0.9993 in the illustrative embodiments. It is also recognized that this value is too high for the exemplary task of locating pulmonary nodules/lesions, as it results in unacceptable levels of false positives although it yielded a high true positive rates.

(b) Data augmentation (step 420): To increase the generalization performance of the above-described, illustrative segmentation model (i.e. performance on unseen data), the system and method performs randomized data augmentation by zooming (random scale factor between 0.9 and 1.1), translating (random translation factor between [−3, 3] pixels in x and y axes of the image coordinate system), flipping between left and right, and rotating (random rotation factor between [−20, 20] degrees) training slices before inputting the data into the neural network for training. This approach can be similar to standard data augmentation techniques proposed in the literature, or can be modified as appropriate. The data augmentation step can be applied independently and randomly with a probability of (e.g.) 0.3 for each slice in the training batch, for every training iteration. Optionally, the type and/or degree of data augmentation can be applied differently to the ground truth designation versus a training image. For example, rotating or zooming ground truth by (e.g.) a one or two degrees, while leaving the training image intact, can render the resulting system more robust to labelling errors in the ground truth data.

(c) Evaluation (step 430): The original training set is split into training and development sets. The development set is used for hyper-parameter optimization and to evaluate the network performance during training. The illustrative evaluation routine consists of thresholding segmented slices in the development set and then merging them into a 3D image, followed by extracting 3D blobs and comparing them with the ground truth to calculate 3D nodulewise recall and precision values. This evaluation technique enables the system and method to evaluate the performance of the segmentation network in terms of its ability to detect nodules in 3D even though it is trained on 2D slices. Optionally, the data augmentation techniques described above in section (b), which are applied to the training data, can also advantageously be applied to evaluation data. For example, rotating or zooming an image of a suspicious lesion so as to maximize the confidence level in the resulting prediction. Note, as used herein, the term “test data” refers generally to a dataset that typically comprises evaluation data or the actual real-life data that the algorithm(s) of the system and method has/have not been exposed to or processed during training. This differs from “training image” or “training data”.

(d) Bayesian inference (step 440): Bayesian inference enables the system and method to obtain the uncertainty, that is, the statistics, associated with model predictions. It is recognized that fusing of the original image with its task-specific summary statistics to be used in subsequent tasks is a novel technique, which has been unavailable in medical imaging or in other computer vision domains. Notably, the technique of the present embodiment differs from other multistage deep learning models in that it effectively captures and propagates model uncertainty from one model to the next in the pipeline by fusing conditional distribution statistics with the original data. By way of further detail, traditional neural networks used for classification/segmentation tasks provide deterministic outputs between [0, 1] during inference to denote the classification probability for a given input image. This approach does not provide model uncertainty, (i.e. “model confidence”). Therefore, the end user (or the next stage of automated analysis) has no useful indication as to whether the model has confidence in its predictions or not for a given text CT scan. Note that it is possible to employ stochastic dropout during testing (i.e. during prediction), which corresponds to Bernoulli variational inference on Bayesian neural networks.

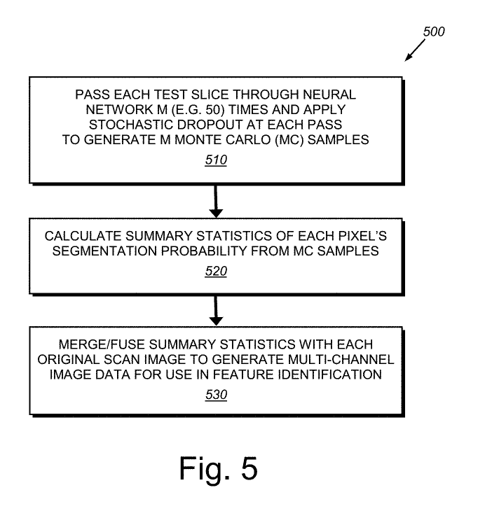

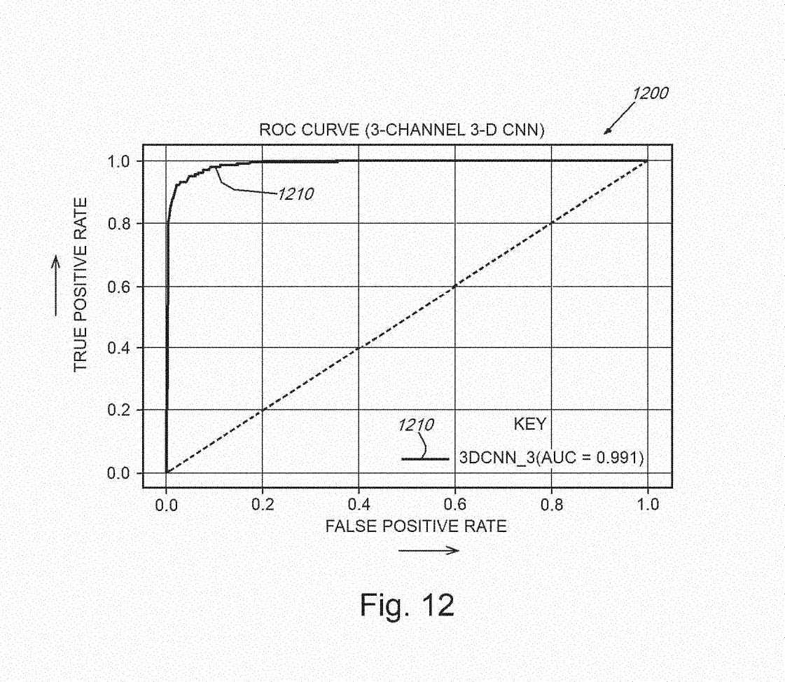

The present embodiment, includes a novel procedure (500 in FIG. 5) that extends this concept in that, during prediction, the system and method passes the test slice through the neural network M (e.g. 50) times (iterations), and applies stochastic dropout at each pass (drop feature maps randomly with a probability of 0.5) resulting in 50 Monte Carlo (MC) samples from the segmentation output probability distribution (step 510). Using these MC samples, the process then calculates summary statistics (mean and variance) of each pixel's segmentation probability distribution, and creates mean and variance segmentation images (step 520). Finally, in step 530, the process of the system and method merges/fuses these summary statistics images with the original CT scan images resulting in three channel CT images (230 in FIG. 2). Note that original CT images are gray scale (having only one channel). Adding extra information from the segmentation model to create additional channels improves the final nodule/lesion detection algorithm as described further below. Also as described below (see graph 1200 of FIG. 12), the illustrative process results in almost perfect nodule/lesion identification performance in the final stage 360 in FIG. 3 of the architecture 300.

Note that confidence levels herein can be characterized by sparse (compressed) representations and/or hierarchical representations. For example, the confidence level can be characterized as a quadtree (also termed quad-tree, which can be used herein to characterize two-dimensions) that those of skill recognize as a tree data structure in which each internal node has exactly four children. Quadtrees are the two-dimensional analog of octrees (also termed oct-tree, which can be used herein to characterize three dimensions), and are most often used to partition a two-dimensional space by recursively subdividing it into four quadrants or regions. The data associated with a leaf cell within the tree varies by application, but the leaf cell represents a unit of interesting spatial information. Likewise, an octree is particularly defined as a tree data structure in which each internal node has exactly eight children. Octrees are most often used to partition a three-dimensional space by recursively subdividing it into eight octants. Similarly, the confidence level can be characterized as a multi-scale image representation, or as a phase representation. By way of useful background information, a hierarchical image representation is described in Burt, P., and Adelson, E, The Laplacian Pyramid as a Compact Image Code, IEEE Trans. Comm. 31, 4, 532-540 (1983).

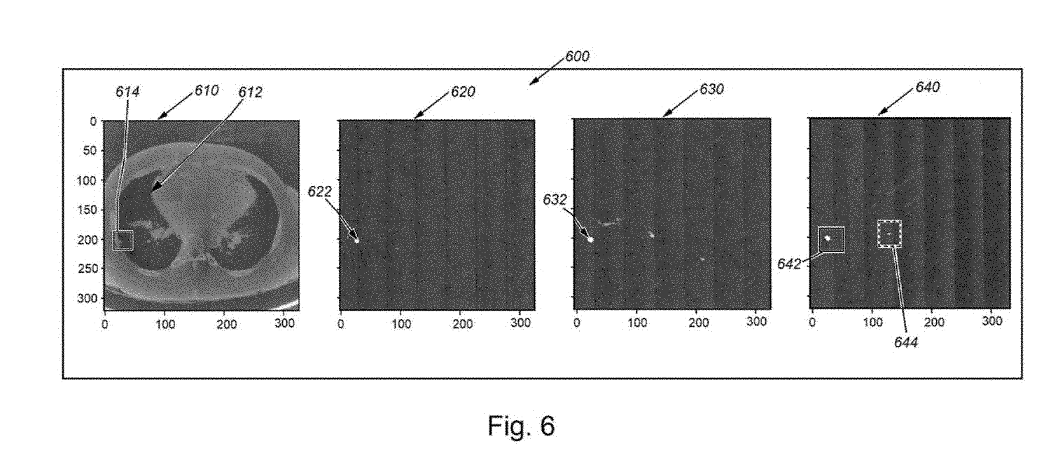

An example of segmentation results is shown in FIGS. 6-8. FIG. 6 demonstrates an example of segmentation on a test image, showing the output of a traditional network where the final thresholded binary image (640 below) has one true positive and one false positive nodule candidate. As shown, the first, leftmost image frame 610 shows a preprocessed (step 310 in FIG. 3), 2D representation of a slice in which an organ (e.g. the patient's lung) 612. The interior wall of the lung 612 includes a small inward protuberance, highlighted by a box 614. Segmentation example on a test image. The second frame to the right 620 shows the results of ground truth nodule segmentation—mainly a small light dot 622 in a uniformly dark field. The next frame to the right 630 shows a segmentation probability map from standard (non-Bayesian) implementation of the segmentation network. The dot 632 is still visible in a dark field, but a number of smaller dots are also distributed around the field. Finally, the rightmost frame 640 shows a thresholded probability map according to the process of the system and method. In this example, there is one true positive enclosed with a box 642 and one false positive enclosed with a dashed box 644.

Note that the exemplary images used herein are made freely publicly available, under license terms that are complied with herein, from The Cancer Imaging Archive via the World Wide Web at URL address https://wiki.cancerimagingarchive.net/display/Public/LIDC-IDRI as part of the The Lung Image Database Consortium image collection (LIDC-IDRI), which consists of diagnostic and lung cancer screening thoracic computed tomography (CT) scans with marked-up annotated lesions. It is a web-accessible international resource for development, training, and evaluation of computer-assisted diagnostic (CAD) methods for lung cancer detection and diagnosis. Initiated by the National Cancer Institute (NCI), further advanced by the Foundation for the National Institutes of Health (FNIH), and accompanied by the Food and Drug Administration (FDA) through active participation, this public-private partnership demonstrates the success of a consortium founded on a consensus-based process. Seven academic centers and eight medical imaging companies collaborated to create this data set which contains 1018 cases. Each subject includes images from a clinical thoracic CT scan and an associated XML file that records the results of a two-phase image annotation process performed by four experienced thoracic radiologists. In the initial blinded-read phase, each radiologist independently reviewed each CT scan and marked lesions belonging to one of three categories (“nodule > or =3 mm,” “nodule <3 mm,” and “non-nodule or =3 mm”). In the subsequent unblinded-read phase, each radiologist independently reviewed their own marks along with the anonymized marks of the three other radiologists to render a final opinion. The goal of this process was to identify as completely as possible all lung nodules in each CT scan without requiring forced consensus. These images have been modified as illustrated herein, and as described. Further required citations include: Data Citation—Armato III, Samuel McLennan. Geoffrey, Bidaut, Luc, McNitt-Gray, Michael F., Meyer, Charles R., Reeves, Anthony P., . . . Clarke. Laurence P. (2015). Data From LIDC-IDRI The Cancer Imaging Archive http://dio.org/10.7937/K9/TCIA.2015.LO9QL9SX; Publication Citation—Armato S G III, McLennan G, Bidaut L, McNitt-Gray M F, Meyer C R, Reeves A P, Zhao B, Aberle D R, Henschke C I, Hoffman E A, Kazerooni E A, MacMahon H, van Beek E J R, Yankelevitz D, et al.: The Lung Image Database Consortium (LIDC) and image Database Resource Initiative (IDRI): A completed reference database of lung nodules on CT scans. Medical Physics, 38: 915-931, 2011; TCIA Citation—Clark K Vendt B, Smith K, Freymann J, Kirby J, Koppel P, Moore S, Phillips S, Moffitt D, Pringle M, Tarbox L, Prior F., The Cancer Imaging Archive (TCIA): Maintaining and Operating a Public Information Repository, Journal of Digital Imaging, Volume 26, Number 6, December, 2013, pp 1045-1057. The authors herein acknowledge the National Cancer Institute and the Foundation for the National Institutes of Health, and their critical role in the creation of the free publicly available LIDC/IDRI Database used in this study.

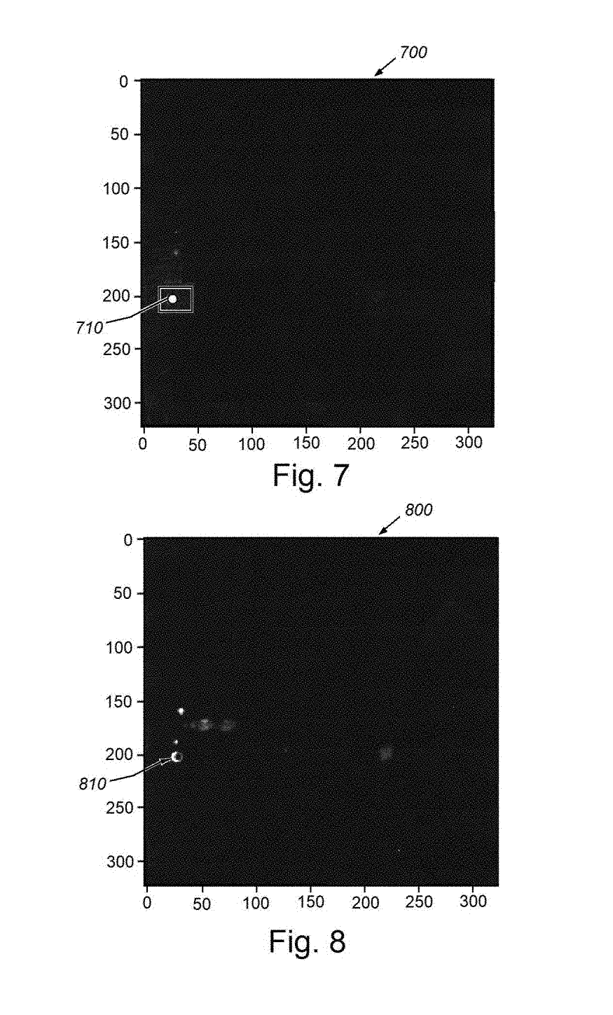

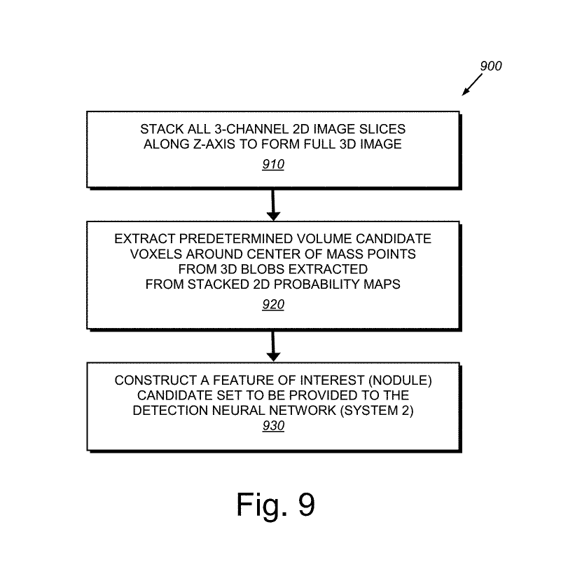

According to FIGS. 7 and 8, the same test image (610 in FIG. 6) is passed through the processes of the illustrative Bayesian neural network herein, and the mean and variance segmentation outputs are presented. FIG. 7 particularly shows an image 700 characterizing the segmentation probability mean. A true positive of the nodule described above is enclosed with a box 710. From this image 700, it is clear that the false positive 644 (of FIG. 6) is no longer visible. FIG. 8 shows an image 800 characterizing the segmentation probability variance. Note that the variance inside the nodule 810 is much lower than the variance on the nodule border, indicating higher uncertainty around the border. This means that the neural network is confident that the nodule interior pixels belong to a nodule, whereas it is not confident about the border. It is clear from FIGS. 7 and 8 that the false positive of the traditional network has been eliminated by the illustrative Bayesian network. Furthermore, the Bayesian process provides pixel-wise model confidence/uncertainty information (shown as segmentation probability variance map in FIG. 8) indicating that it is highly confident on where the true nodule is located, and not as highly confident on nodule borders and a few other locations in the image.

3. Post-processing and 3D Voxel Extraction 340, which, after passing CT images through the segmentation network (e.g.) 50 times (i.e. 50 MC runs), computes the average of these runs to obtain the mean segmentation probability maps. The process 340 then stacks these 2D probability maps along z-axis to form 3D segmentation mean probability maps. These probability maps are then thresholded, 3D blobs are extracted, and the center of mass of these blobs is calculated. These center of mass points construct the candidate set for the next stage of the process. The threshold is optimized using a development set, (for example, by maximizing recall and precision on a development set). After obtaining center of mass points, 3D voxels (or nodule candidates) are extracted for each 3D CT scan. This can occur according to the following procedure: All three channel 2D image slices (the output of process 330) are stacked—where each slice has three channels corresponding to the original image, the segmentation probability mean map, and the segmentation probability variance map—along the z-axis to form a full 3D image. Then 32 mm³ candidate voxels are extracted around the center of mass points of the blobs described above. The voxels construct the nodule candidate set that are provided to the nodule detection neural network in the CADe machine learning pipeline (System 2 (240) in FIG. 2). Note that the above-described segmentation process 330 and the post-processing/3D voxel extraction processes collectively comprise the localization module 350 of the overall system and method architecture 300.

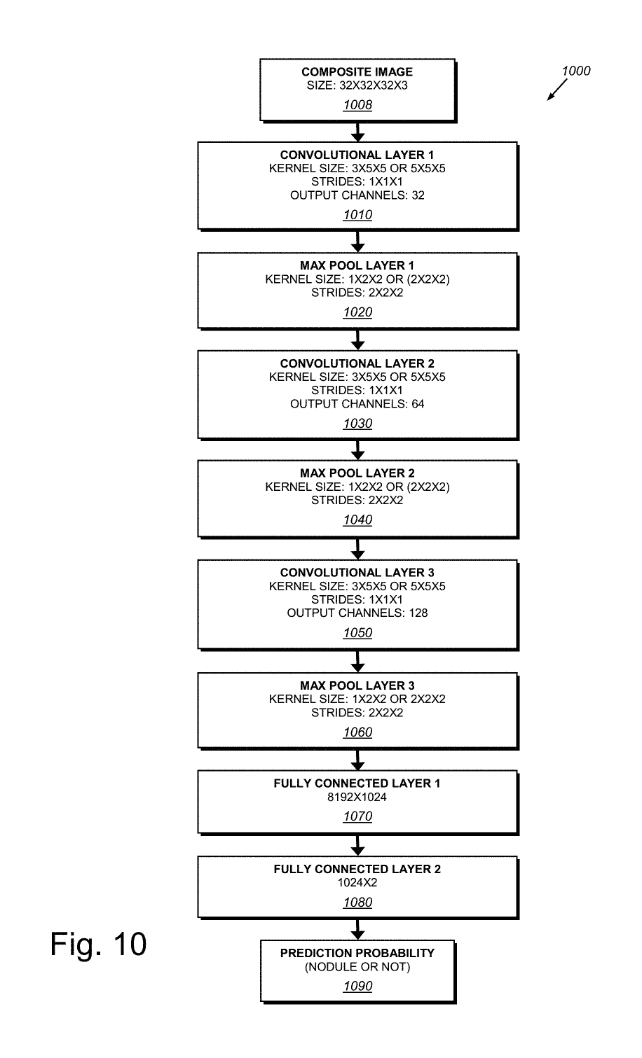

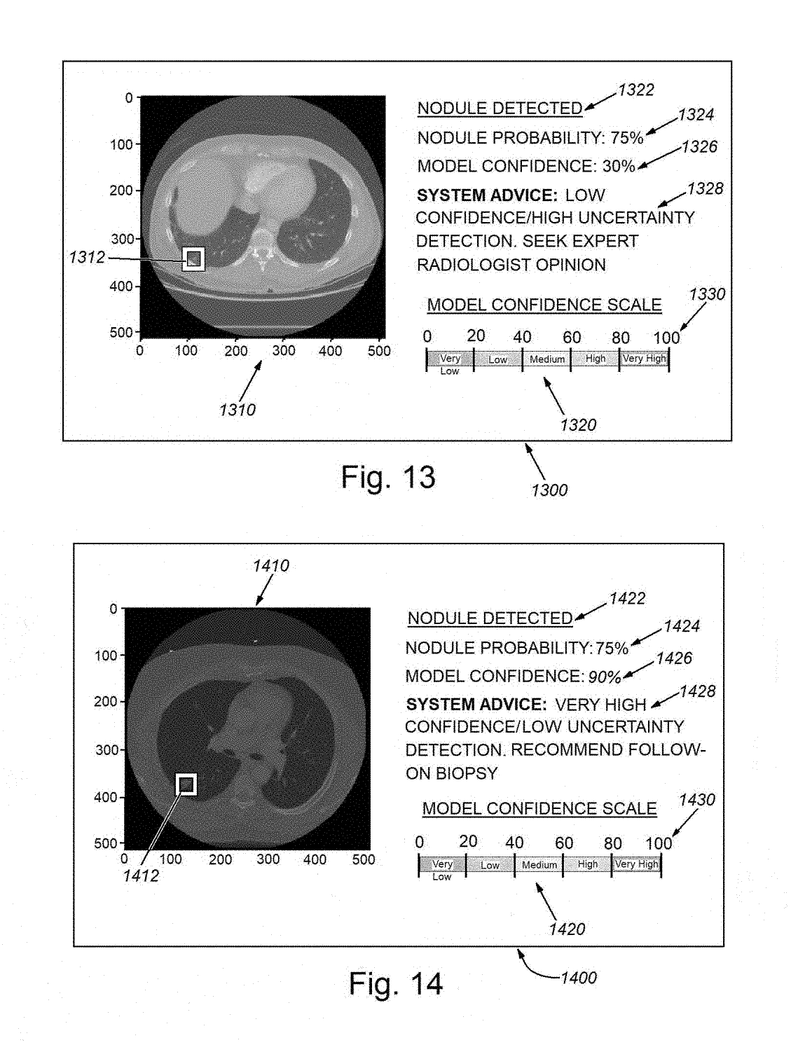

4. Nodule Identification 360, in which the goal of this stage in the overall architecture 300 is to reduce false alarms while maximizing the probability of detection of nodules (results including nodule locations and e.g. scores 370). As described above, the candidates are of the size 32×32×32×3 where the first three dimensions are (e.g.) millimeters and the last dimension is the number of channels corresponding to the original CT scan, along with the mean and variance images obtained from the probabilistic segmentation outputs (from the Bayesian neural network). Before inputting these voxels into the next machine learning system for training, they are preprocessed by normalizing each voxel to have zero mean and unit variance across all channels (i.e. on a channel-by-channel basis) in the training dataset. For the final identification task as shown in the process block 360, the system and method provides a novel 3D convolutional neural network (CNN) architecture 1000, depicted in FIG. 10, which processes input 32×32×32×3 composite images 1008. The 3D CNN of this exemplary embodiment has three convolutional layers (1, 2 and 3) 1010, 1030 and 1050, respectively, each followed by three max pooling layers (1, 2 and 3) 1020, 1040 and 1050, respectively. These are all followed by two fully connected layers (1 and 2) 1070 and 1080. In experimenting with different kernel sizes for the convolutional layers it has been recognized that sizes 3×5×5, 5×5×5, and 3×3×3 (in z-y-x axes) worked well for the particular identification task. The procedure 1000 employs identical kernel sizes across all convolutional layers in this example. The first convolutional layer 1010 thereby outputs 32 channels, resulting in 32 32 mm³ feature maps. The second and third convolutional layers 1030 and 1050 each output 64 and 128 feature maps, respectively. In the max pooling layers, 1×2×2 or 2×2×2 windows are used, with strides of 2×2×2, thereby reducing the size of the input feature maps by half. For example, the output of the first max pooling layer is 32 16 mm³ feature maps. In addition to what is shown in FIG. 10, the system and method can also apply stochastic dropout (with probability 0.5) in the fully connected layers 1070 and 1080 for training. Optionally, it is contemplated that an additional dropout can be added after the convolutional layers 1010, 1030 and 1050—which is a similar approach to the above-described technique in the segmentation neural network 220). Note that such dropout layers enable the system to perform stochastic dropout during test (and/or evaluation) time to obtain a final nodule detection output (nodule probability) with an associated confidence level (which is similar to the segmentation neural network 220). This detection result and associated confidence level is then presented to the end user. This Bayesian technique not only improves the final detection performance, but it also provides a model confidence level to the end user (see FIGS. 13 and 14, by way of example). Additionally, for training of the 3D CNN, data augmentation occurs, similarly to that performed for the above-described segmentation network. Data augmentation is particularly helpful for the positive class (i.e. for true nodule candidates) as they are typically highly underrepresented in the candidate set (i.e. the ratio of the number of positive class data samples to the number of negative class data samples is very low). The randomized data augmentation routines consist of zooming (random scale factor between 0.9-1.1), translation (random translation factor between [−3, 3] pixels in x-y-z axes), rotations of random multiples of 90 degrees, and random rotations by a factor between [−20, 20] degrees. Each data augmentation step is applied independently and randomly with a probability of 0.5 for each voxel in the training batch. To solve the class imbalance problem, a different strategy is employed from that applied to the segmentation network (220). Thus, instead of using a weighted cross entropy as the training cost function that is adopted for the segmentation network (see above), in this stage, the standard (unweighted) cross entropy cost function is employed as follows:

$${ℒ}_{B} ( θ ) = {\sum }_{i ∈ B} y ( i ) log \hat{y} ( i ; θ ) + ( 1 - y ( i ) ) log ( 1 - \hat{y} ( i ; θ ) )$$

where y(i)∈{0, 1} denotes whether a voxel (or a nodule candidate) is a nodule (1) or not (0) for voxel i, ŷ(i;θ)∈[0, 1] is the output of the network (1090 in FIG. 10), denoting the probability that voxel i is a nodule (parameterized by θ), θ is the vector of network weights that are learned via training (i.e. by minimizing the loss function L(θ)), and B is the set of voxels in a training batch. To deal with class imbalance, it is ensured that each training batch has approximately the same number of positive and training examples/voxels. This can be achieved by randomly choosing between each class with a probability of 0.5 to input into a training batch. Although this technique results in the neural network having to process through the entire set of positive examples significantly more frequently than that of negative examples, data augmentation assists the network in alleviating overfitting for the positive class.

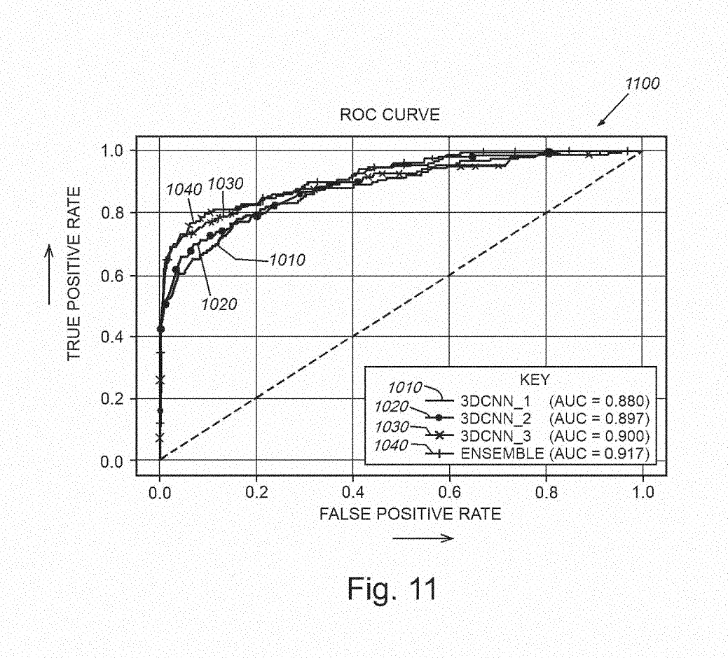

An example nodule identification results is shown in the graphs 1100 and 1200 of FIGS. 11 and 12, respectively, which plot the true positive rate of identification versus the false positive rate. FIG. 11 illustrates the case where the original single-channel CT image is used exclusively for voxel classification without (free-of) normalization of the voxel intensities. This graph 1100 thereby depicts results from three discrete 3D CNNs, where the difference between curves 1110, 1120 and 1130 (area under curve (AUC) of 0.880, 0.897, 0.900, respectively) is from different kernel sizes used in convolutional and max pooling layers (refer to architecture 1000 of FIG. 10). The graph 1100 also plots a curve 1140 with results of an ensembling technique in which the system and method averages the outputs of the three CNNs (1110, 1120 and 1130). It has been recognized that ensembling offers a slight improvement in the performance of the identification procedure. Note that an alternative to performing ensembling via an averaging technique, can include ensembling via a voting scheme or unanimity requirement. For example, the procedure refrains from identifying a structure in an image as a nodule/lesion unless all three (3) learning networks agree that it is—particularly in implementations of the system in which a priority is to minimize false positives.

The system and method then fuses the original CT image with the Bayesian segmentation network output images (mean and variance images), normalizes the voxel intensity values, and runs this structure through one of the 3D CNNs. The result is shown as the plot 1210 in FIG. 12. It is clear that for this specific set of images and optimized parameters, that the procedure approaches perfect (1.0 or 100%) true positive nodule identification result (actual area under the ROC curve of 0.991 where a perfect score would be 1.0). The improvement over the case where a non-Bayesian non-normalized single-channel CT image is used is significant (from 0.90 to 0.991). It is clear that the illustrative Bayesian technique of the system and method provides model confidence values, a highly useful functionality for the end-users in clinical settings. Additionally, the illustrative technique also improves the overall performance of an exemplary CADe system with appropriately optimized parameterization.

### III. User Interface and Results

The above-described system and method uniquely provides a technique for propagating and fusing uncertainty in a multi-stage deep learning pipeline for computer vision applications. A highly significant advantage of this solution is to provide and propagate model confidence, which is lacking from other multi-stage deep learning models, including other CADe solutions. Model confidence information is highly important for critical decision making applications, such as cancer detection, which renders the overall diagnostic system more interpretable and easier to adopt by and gain the trust of practitioners (e.g. doctors and radiologists). By way of example, FIGS. 13 and 14 show two graphical user interface (GUI) displays 1300 and 1400, which can be employed as outputs in a nodule identification embodiment in which the underlying CADe model detects a nodule and presents the location of the nodule to the end-user (e.g. a radiology technician or a physician) along with a confidence score for this (fully) automated prediction. In the exemplary display 1300 of FIG. 13, an image 1310, with appropriate x-axis and y-axis scaling (e.g. millimeters, etc.), of a slice of the region of interest (lung) is displayed in a box to the left. A highlighted box 1312 is displayed in the region of interest/concern where a candidate nodule/lesion is located by the CADe system. This image 1310 is accompanied by textual and graphical information in the right hand column 1320 of the display 1300. This information includes the status of nodule detection 1322 (in this example, a nodule is shown as DETECTED by the CADe system); the probability that the nodule or lesion is an actual cancerous nodule of concern 1324 (in this example, the model predicts that the lesion has 75% chance of being a nodule); and the confidence of this prediction 1326 (in this it has low confidence (30%) in its prediction) and color coded advice 1328, which in this example advises or suggests the user (e.g. a radiology technician or radiologist) to seek expert opinion (e.g. a radiologist) on further diagnosis due to the low confidence score. An optional color-coded confidence scale 1330 maps to the advice text 1328 to further accentuate the required action to the user. More particularly, the depicted low confidence can occur in scenarios where the model has insufficient training with examples similar to that particular nodule located in this runtime operation. Alternatively, the model may be unfamiliar with images from that particular CT scanner (for example, during training). The system therefore warns the user that (due to insufficient training data) it cannot provide a confident decision, and encourages the user to seek an expert opinion (for example, a more-credentialed/experienced practitioner, or a second opinion). In other words, the model knows what it does not know, which is a functionality particularly lacking in current automatic/automated CADe systems.

In the exemplary display 1400 of FIG. 14, the image 1410 is again displayed on the left and a box 1412 is placed by the system around the candidate nodule/lesion. The right hand column 1420 contains status and advisory text and graphics, including detection status 1422 (e.g. nodule DETECTED); nodule prediction probability 1426 (e.g. 75%—similar to the previous example); model confidence 1426 (e.g. 90%—which is a relatively high value compared with the example of display 1300 above); and system advice 1428, which is color-coded with high confidence based on the scale 1430. In this example, the prompt to the user is to suggest/advise the desirability of a follow-on invasive test (e.g. a biopsy), and/or prompt an expert (e.g. the radiologist) to take such further investigative action (e.g. use of CADx techniques) on a high-priority basis, as there is clear evidence of a nodule/lesion that is likely cancerous. It should be clear that such an automated system generally improves accuracy of diagnosis, and speeds/improves both treatment decisions and outcomes.

### IV. Alternate Applications

As described above, the system and method herein can be applied to a variety of 2D and 3D datasets derived from a variety of types of sensing and/or acquisition devices, and based on a variety of sensing media and transmitters/emitters of such. In the medical and diagnostic arts, in addition to the exemplary CT scanner and CT scan slices, devices can include MRI devices and imagery, images, and/or targeted contrast ultrasound images of human tissue (e.g. microbubble agent, etc.), and the subject or target to be identified is/are potentially cancerous lesion(s). More generally, the system and method herein is applicable to providing solutions that account for potential unreliability in CADe, and can estimate or predict the degree of such unreliability. In further embodiments, the system and method can apply to 2D and 3D data that is derived from automotive sensors and sensor arrays (for example as used in collision avoidance, self-parking and self-driving arrangements). Such sensors can include visible light cameras and pattern-recognition, LIDAR and/or RADAR and the resulting images are used by the automotive processor(s) to evaluate include obstacles to avoid, street signs to identify, traffic signals, road markings, and/or other driving hazards. More generally, the system and method herein is applicable where uncertainty information is fused temporally across multiple frames to refine previously computed confidence estimates. For example, if an object of interest is a pedestrian with high confidence in acquired 2D or 3D image frames 1-3 and 5-8 of a stream of acquired images (by any of the devices/imaging modalities described above), there is a high likelihood that the object is a person in frame 4 as well. Even if temporary occlusions or lighting changes in frame 4 (shadows, flying birds, camera glare, fog, haze, smoke, dust, etc.) cause uncertainty when frame 4 is evaluated in isolation. In general, the system and method can apply where the temporally fusing the confidence estimate is based on subject/object tracking (i.e. a moving object, a moving acquisition device (aircraft, satellite, watercraft, robot manipulator, conveyor, etc.) or both), rather than purely upon spatial location. The fusion of such confidence information can occur in a variety of ways. For example, the system and method can employ (a) the MAXIMUM across multiple time points (e.g. if the system ever identifies a bird, that means a bird is there); (b) the MINIMUM across multiple time points (e.g. unless it always appears as a tumor, do not advise surgery); (c) the MEAN across multiple time points; and/or (d) rejection of extreme outliers across multiple time points—for example, by applying regression or other model-fitting techniques to the confidence data or to the combination of confidence and intensity data. In further embodiments, the data to be evaluated can be based on aerial acquisition and the target property to be identified is a sea surface anomaly (ship's wake, submerged watercraft's signature, such as a Bernoulli hump, obstacle to navigation, etc.), or the target property to be identified is an aerial property such as a storm system, cloud pattern, aircraft/spacecraft and/or its exhaust heat plume or contrail, and/or animal (bird migration patterns, etc.).

In further embodiments, the interface presented to the user, and/or the modality for doing so, can be highly variable and can include 2D or 3D image displays, virtual reality viewers, printed 2D images, 3D-printed shapes and a wide range of multi-media presentations with various tags, flags, control and input screen objects in a variety of colors, shapes, etc. More generally, the confidence level information about objects classified by machine learning networks to an end-user (human or computational) can include a spatial indicator that augments the ordinary signal intensity in a manner that conveys certainty. For example, a color change, highlighting, line width change, imposition of a texture, 3D embossing of a printed map, etc. can be used to convey identified features.

The system and method can provide processes in which one or more acquired images are scanned for presence of a target property, in which a first set of images is acquired and analyzed to detect that property, and a confidence level associated with that detection is determined. This is followed by iteratively adjusting one or more image acquisition parameter(s) (e.g. camera focus, exposure time, X-ray/RADAR/SONAR/LIDAR power level, frame rate, etc.) in a manner that optimizes/enhances the confidence level associated with detection of the property of interest. Generally, the system and method can detect a property of interest in a sequence of acquired images, in which image interpretation parameter(s) (such as image thresholding levels, image pre-processing parameters such as multi-pixel fusion, image smoothing parameters, contrast enhancement parameters, image sharpening parameters, machine learning decision-making thresholds, etc.) is/are iteratively adjusted so as to optimize the confidence level in detection of the desired property.

Note that in further embodiments, the system process(or) can be arranged with a morphological filter that adjusts a confidence level associated with a region based on confidence levels associated with neighbors of the region. This helps to overcome certain limitations of uncertainty measurement.

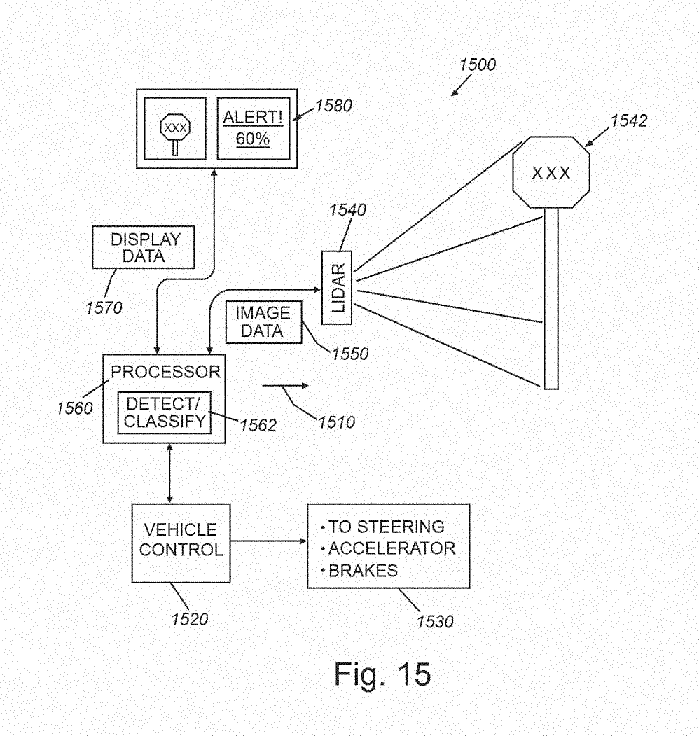

In additional embodiments signals acquired from various sensing modalities, such as RADAR, LIDAR and/or ultrasound, can be used to control an action or operation on a device (e.g. a land vehicle, aircraft or watercraft) where an object classifier (derived based upon the illustrative processes herein) reports a low confidence level in its classification of that object (e.g. an obstacle, sign, other vehicle, etc.). For example, if the external condition is so occluded (e.g. foggy) that an object cannot be recognized (e.g. is it a stop sign, person, etc.?) then the device controller instructs a subsystem to change operation—for example, shut off cruise control and/or apply brakes and/or decelerate. An exemplary vehicle-based detection and characterization arrangement 1500 is shown schematically in the diagram of FIG. 15. In this example, the vehicle (e.g. a land vehicle (e.g. a car or truck)—but the principles apply variously to water vehicles (boats, submersibles, etc.) or aerial vehicles (e.g. fixed wing, rotary wing, drone, etc.), is shown moving (arrow 1510) toward a sign or other critical structure for which decisions are required either by (e.g.) the autonomous vehicle control system, the driver, or both. The control system in this example provides input to and feedback from steering, throttle and braking systems 1530. A LIDAR 1540 and/or other form of sensor described above senses an area (e.g. the forward region of the vehicle) continuously, or when called upon—for example when triggered by other sensors or the driver. In this example, an exemplary road sign 1542 has come into range. The information from the LIDAR is delivered as 3D imagery 1550 to a processor that includes an object detection and classification process(or)/module 1552 that operates in accordance with the teachings of the embodiments herein (refer also below). The object is thereby detected, classified and such information is used by other logic (for example, within the processor 1560) and/or vehicle control 1520 to determine whether control operations are required. Similarly, the details of the object along with confidence or other validating information 1570 can be transmitted to a vehicle display 1580 for the driver or other interested party to observe and (if necessary) act upon. Data can also be stored in an appropriate storage device and/or transmitted via a wireless link to a remote site.

In an exemplary LIDAR system as described in FIG. 15 with a non-uniform scan control system, a workflow may include a detection stage and a classification stage (similar to the above-described CADe methodology). Using detection statistics for the sensing mechanism (e.g. Asynchronous Geiger Mode Avalanche Photodiode), a revisit priority queue can be managed using uncertainty. Additionally, the uncertainty of object detection can be passed to the object classification stage, and can further modify the revisit priority queue based upon model uncertainty of the object classifier. By tracking the uncertainty in a per-voxel method, the revisit decision can also determine the size of the area to be revisited (either expand or contract depending on the size of the region of uncertainty).

In one operational example, at further distances, the signal-to-noise ratio (SNR) is reduced, and the confidence of detection is generally lower, and the spatial bounds less certain. If a low confidence detection is found in a priority area such as directly in the path of the autonomous vehicle it may trigger a revisit of the area around the detection to determine if the detection is real or spurious. In another operational example the confidence in the object classification is within an area of interest. Object classification in low priority areas (e.g. the side of the vehicle) may not trigger an immediate revisit, but a low confidence classification in the front of the vehicle may trigger a revisit to drive down the uncertainty of the object classification. As shown in FIG. 15, both of these examples also apply for directing the user's (drivers) attention through display visualization (which can be similar to the displaying of uncertainty in the depicted CADe GUI of FIGS. 13 and 14), or as triggering a control action such as transferring control of and autonomous vehicle back to the user/driver and/or applying immediate action, such as braking.

In further embodiments, it is contemplated that the acquisition device, which performs data acquisition is an ad-hoc sensor network, in which the network configuration (tasking) is reconfigured so as to optimize confidence level in the detected parameter—for example, a network of acoustic sensors in which the local data fusion (communication) is adjusted based on the confidence of detection of a signal of interest.

### V. Conclusion

It should be clear that the above-described system and method for applying and quantifying uncertainty in reasoning related to 2D and 3D spatial (image) features provides a more effective model for predicting the presence or absence of certain feature sets. This technique is particularly advantageous in the area of medical imaging, but has broad applicability to a wide range of possible applications that employ image data or other datasets that exhibit similar characteristics. The data can be presented to users in a variety of ways using a variety of display modalities, which can be best suited to the decision making requirements of the user.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention. For example, as used herein, various directional and orientational terms (and grammatical variations thereof) such as “vertical”, “horizontal”, “up”, “down”, “bottom”, “top”, “side”, “front”, “rear”, “left”, “right”, “forward”, “rearward”, and the like, are used only as relative conventions and not as absolute orientations with respect to a fixed coordinate system, such as the acting direction of gravity. Additionally, where the term “substantially” or “approximately” is employed with respect to a given measurement, value or characteristic, it refers to a quantity that is within a normal operating range to achieve desired results, but that includes some variability due to inherent inaccuracy and error within the allowed tolerances (e.g. 1-2%) of the system. Note also, as used herein the terms “process” and/or “processor” should be taken broadly to include a variety of electronic hardware and/or software based functions and components. Moreover, a depicted process or processor can be combined with other processes and/or processors or divided into various sub-processes or processors. Such sub-processes and/or sub-processors can be variously combined according to embodiments herein. Likewise, it is expressly contemplated that any function, process and/or processor herein can be implemented using electronic hardware, software consisting of a non-transitory computer-readable medium of program instructions, or a combination of hardware and software. Additionally, in alternate embodiments, it is contemplated that some of the multi-stage machine learning models herein can be combined into a single model. For example, the CADe problem can be approached as an object detection problem in 3D and could potentially be solved by using regional convolutional neural networks (RCNNs) to detect candidate nodule locations. While, standard RCCNs do not capture epistemic (model) uncertainty, such can be modified methods (similarly to the above-described techniques) to modify them into Bayesian models. Accordingly, this description is meant to be taken only by way of example, and not to otherwise limit the scope of this invention.

What is claimed is:

1. A method for detecting and/or characterizing a property of interest in a multi-dimensional space comprising the steps of:
receiving a signal based upon acquired data from a subject or object in the multi-dimensional space;
interpreting a combination of information from the signal and confidence level information; and
based on the interpreting step, performing at least one of detection and characterization of at least one property of interest related to the object or subject.

2. The method as set forth in claim 1 wherein at least one of (a) the multi-dimensional space is a 2D image or a 3D spatial representation, (b) the at least one of detection and characterization includes use of a learning algorithm trained on the combination of information from the signal and confidence level information, and (c) the at least one of detection and characterization includes evaluation by a learning algorithm that has been trained according to step (b).

3. The method as set forth in claim 1, further comprising estimating the confidence level based upon uncertainty using dimensional representations of a lower dimension than the multi-dimensional space, in which at least two estimates of the uncertainty based on the dimensional representations of the lower dimension are assembled to form a representation of the uncertainty in the multi-dimensional space.

4. The method as set forth in claim 1, further comprising estimating the confidence level based upon uncertainty, in which a degree of the uncertainty is modeled on a comparable spatial scale to an intensity of the signal.

5. The method as set forth in claim 4 wherein the step of estimating includes using additional image channels to represent each of a plurality of confidence levels.

6. The method as set forth in claim 1 in which the confidence level is represented by at least one of a sparse representation and a hierarchical representation.

7. The method as set forth in claim 6 wherein the confidence level is represented by at least one of a quadtree for two dimensions, an octree for three dimensions, a multi-scale image representation, and a phase representation.

8. The method as set forth in claim 1 wherein the acquired data is vehicle sensor data, including at least one of LIDAR, RADAR and ultrasound that characterizes at least one object in images to evaluate, including at least one of (a) obstacles to avoid, (b) street signs to identify, (c) traffic signals, (d) road markings, and/or (e) other driving hazards.

9. The method as set forth in claim 8 further comprising controlling an action or operation of a device of a land vehicle, aircraft or watercraft based on an object classifier that reports low confidence level in a classification thereof.

10. The method as set forth in claim 1 wherein the acquired data is medical image data, including at least one of CT scan images, MRI images, or targeted contrast ultrasound images of human tissue and the property of interest is a potentially cancerous lesion.

11. The method as set forth in claim 1 wherein the detection and characterization is diagnosis of a disease type, and the information from the signal is one or more suspected lesion location regions-of-interest and the confidence levels are associated with each region-of-interest that is suspected.

12. The method as set forth in claim 1 wherein the steps of receiving, interpreting and performing are performed in association with a deep learning network that defines a U-net style architecture.

13. The method as set forth in claim 12 wherein the deep learning network incorporates a Bayesian machine learning network.

14. The method as set forth in claim 1 wherein the acquired data is received by an ad-hoc sensor network, in which a network configuration is reconfigured so as to optimize the confidence level in a detected parameter.

15. The method as set forth in claim 14 wherein the sensor network includes a network of acoustic sensors in which a local data fusion is adjusted based on a confidence of detection of a property of interest in the signal thereof.

16. The method as set forth in claim 1 wherein the confidence level is associated with the signal to enhance performance by using a thresholding step to eliminate low confidence results.

17. The method as set forth in claim 1 wherein the signal is based on aerial acquisition and the property of interest is related to a sea surface anomaly, an aerial property or an air vehicle.

18. The method as set forth in claim 1 wherein the confidence level related to the subject is classified by machine learning networks to an end-user, including a spatial indicator that augments an ordinary intensity of the signal in a manner that conveys certainty.

19. The method as set forth in claim 1 further comprising fusing uncertainty information temporally across multiple image frames derived from the signal to refine an estimate of the confidence level.

20. The method as set forth in claim 19 wherein the step of fusing is based on at least one of tracking of the subject and spatial location of the subject.

21. The method as set forth in claim 20 wherein the step of fusing includes (a) taking a MAXIMUM across multiple time points (b) taking a MINIMUM across multiple time points, (c) taking a MEAN across multiple time points, and (d) rejecting extreme outliers across multiple time points.

22. A system that overcomes limitations of uncertainty measurement comprising:
a morphological filter that adjusts a confidence level associated with a region based on confidence levels associated with neighbors of the region.

23. A method for acquiring one or more images to be scanned for presence of a property of interest comprising the steps of:
acquiring a first set of images;
analyzing the first set of images to detect the property of interest and a confidence level associated with the detection;
iteratively adjusting at least one image acquisition parameter in a manner that optimizes or enhances the confidence level associated with the detection of the property of interest.

24. A system for detecting a property of interest in a sequence of acquired images comprising, in which at least one of a plurality of available image interpretation parameters is iteratively adjusted by a processor so as to optimize a confidence level in detection of the property of interest.

25. The system as set forth in claim 24 wherein the image interpretation parameters include at least one of image thresholding levels, image pre-processing parameters, multi-pixel fusion, image smoothing parameters, contrast enhancement parameters, image sharpening parameters, and machine learning decision-making thresholds.

26. A system for utilizing a conventionally trained neural network that is free-of training using confidence level data to analyze signal data that has been augmented by confidence level, wherein the signal data is weighted based on confidence prior to presentation to the conventionally-trained neural network.

27. The system as set forth in claim 26 wherein the conventionally trained neural network comprises a tumor lesion characterizer.
